(19) United States
(12) Patent Application Publication
(10) Pub. No.: US 2024/0415227 A1
(43) Pub. Date: Dec. 19, 2024

(54) DIGITALIZED THERAPEUTIC FOOTWEAR

(71) Applicant: KARAN JOGESH BHASIN, MUMBAI (IN)

(72) Inventor: KARAN JOGESH BHASIN, MUMBAI (IN)

(21) Appl. No.: 18/036,668

(22) PCT Filed: Mar. 4, 2023
(86) PCT No.: PCT/IB2023/052031

§ 371 Date: May 12, 2023

(30) Foreign Application Priority Data
Mar. 7, 2022 (IN) 202221012073

(51) Int. Cl. A43B 3/44; A43B 7/00; A61B 5/00

(52) U.S. Cl. CPC A43B 3/44; A43B 7/00; A61B 5/6807; A61B 2562/0219

(57) ABSTRACT

The present invention relates to a digitalized therapeutic footwear for detecting ailments of health system. The footwear has a sensor system containing a plurality of sensors, three zones pressure points having toe zones, middle foot zone and heel zone assess variations in brain, gastrointestinal diseases and enhancing painless long standing hours that the cumulative effects of zones enhances energy level. A communication port connected to sensor system serves for transmitting data on force exerted by the wearer of the footwear that data detected by each sensors in a repository chip which embedded in the footwear which connected external device configured to receive processing the data; Footwear serves the purpose of enhancing health level and detecting ailments of health system as means of early warning system in treating the same.

### FIELD OF THE INVENTION

The present invention relates to a footwear having an inbuilt sensor system. Specifically, the present invention relates to a footwear having an inbuilt sensor system and pressure points affixed around the inner sole of the footwear for the purpose detecting health ailments which can be detected and read on a sensor system connected on to a communication port within the footwear that read on external device.

### BACKGROUND OF THE INVENTION

Numerous shoes and shoe sensor devices have been provided in the prior art. For example, U.S. Pat. Nos. 5,408,873; 5,566,479; 5,619,186; and 5,642,096 all are illustrative of such prior art. While these units may be suitable for the particular purpose to which they address, they would not be as suitable for the purposes of the present invention as heretofore described. In U.S. Pat. No. 6,122,846, the invention disclosed a Force monitoring system that includes a force sensing unit which monitors weight imposed at any point along the length of a wearer's foot within a shoe. Also measures weight imposed on a wearer's foot for each step and reports to the wearer any weight imposed on the wearer's foot exceeding pre-selected upper limits, or weights below pre-selected lower limits. The inventors of the present invention discloses the athletic shoe in which LED glows when the forefoot/heel part of the sole touches the ground based on pressure and bend sensors.

The problem to be solved in the present invention, to provide a kind of health monitoring footwear, in order to determination and analysis plantar pressure and relevant parameter, can obtain some physiology or the pathological information of human body

Thus, there is a need to provide a digitalized therapeutic footwear that can provide complete information with regard to health condition of a person.

### OBJECTS OF THE INVENTION

Object of the present invention is to provide a footwear sensor which is able to provide an early warning system with regard to health condition of a person.

Another object of the present invention is to provide for a device for sensing health fluctuations of the wearer of the footwear.

### SUMMARY OF THE INVENTION

The following presents a simplified summary of the disclosure in order to provide a basic understanding to the reader. This summary is not an extensive overview of the disclosure and it does not identify key or critical elements of the invention or delineate the scope of the invention. Its sole purpose is to present some concepts disclosed herein in a simplified form as a prelude to the more detailed description that is presented later.

The present invention relates to footwear to determine the status of the health through transmission of physiological response to trigger by the pressure points, configured to stimulate the tissues and nerves located at the feet of the wearer. The exchange of the stimulus that received by the sensors fixed at different points inside the Footwear transmits the data to the communication port to convert the received signals to readable format that was displayed in the external device as final output to assess the status of the health.

The sensor unit of footwear detects the health condition of the wearer of the footwear that measures several diagnostic parameters which are related to cardiovascular system, nerves system, digestive system and other systems. The footwear system completely acts as prophylactic measures to keep health of the individual monitored and alarm to proceed for health related treatments. Assessment of physiological readings is essential in assisting further to keep wearer monitoring daily activities that directly affecting heath issues and keep check on the real-time assessment of diagnostic parameters.

In the present invention, the inner sole is responsible for receiving the foot of the wearer. importantly the said inner sole, demarcated into a minimum of three zones each having on them pressure points for purpose of providing the wearer therapeutic comfort.

According to one aspect of the present invention, the GPS tracking devices are embedded in the inner sole of the present invention to track and ensures safety of the wearer.

According to one aspect of the present invention, the present invention is to provide a footwear sensor, particularly suited for diabetics and other related metabolic diseases.

The invention may also relate to any alternative embodiments within the scope of the claims.

### BRIEF DESCRIPTION OF THE DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 illustrates a foot bed of the digitalized therapeutic footwear according to one embodiment of the invention.

FIG. 2 illustrates a sensory board to be inserted in shoe sole according to one embodiment of the invention.

FIG. 3 illustrates a sensor chip 302 present in the sensory board according to one embodiment of the invention.

FIG. 4 illustrates schematic diagram of GNSS module, power module, UART level shift, reset and PSS.

FIG. 5 illustrates schematic diagram of MCU, flash memory and programmer and debugger.

FIG. 6 illustrates schematic diagram of oximeter/heart rate, accelerometer/gyroscope and weight sensor.

### DETAILED DESCRIPTION OF THE INVENTION

It is to be understood that the present disclosure is not limited in its application to the details of composition set forth in the following description. The present disclosure is capable of other embodiments and of being practiced or of being carried out in various ways. Also, it is to be understood that the phraseology and terminology used herein is for the purpose of description and should not be regarded as limiting.

The present invention relates to a digitalized therapeutic footwear having an inbuilt sensor system and pressure points affixed around the inner sole of the footwear for the purpose detecting health abnormalities which can be detected and read on a sensor system connected on to a communication port within the footwear. The communication stored in a chip which meant for the purpose of collecting such data and converting it into a readable format.

In the insole for shoes comprising a first layer on which a plurality is made of projections/pressure points having a predetermined shape and thickness, located in correspondence of sensitive points of the foot sole that are linked to relevant parts of the human body, in accordance with the principles of foot sole reflexology, and capable of massaging said foot sole at said sensitive points, exploiting the regular change of the distribution of the weight of the body between foot tip and heel, said projections being alternatively compressed, absorbing the energy charged by the weight of the body, and then released, returning this energy and thus stimulating the foot sole characterized in that the insole comprises at least one layer of a cushioned elastically yielding material that allows transpiration and has an overall thickness.

Pressure points are simulative instruments that triggers muscles on mobilization of the foot that eases in transmission of signals related to the respective messenger channel in muscular/nerves system that is identified by the sensors located at different positions inside the footwear. The foot pressure on the zones focused for brain, digestive system and pain management on long hours standing position. Pressure points eases stress, pushes levels of energy, reduces anxiety, fatigue, more relaxed, reduced pain feeling as psychological benefits that keeps body in feel better working condition.

Three zones that considered in the invention to accelerate the energy level to keep and trigger mechanism that helps in generating the charges in the body that received by sensors in turn transmit it to the communication port. Working mechanism of the invention involves the multiple triggers from the pressure points that specially designed for the present invention that presses the area that stimulates nerves system in turn sends messages to the Central Nerves system.

The pressure points in the present invention, material used selected from the TPE, TPR, TPU, TPC, TPA, EVA and Any other rubber mouldable in the insole of the footwear. Thermoplastic rubbers are more effective, economic, and beneficial to Environmental protection. Thermoplastic elastomers (TPE), sometimes referred to as thermoplastic rubbers, the ability to stretch to moderate elongations and return to its near original shape having longer life and better physical range than other materials. EVA (Ethylene-vinyl acetate), elastomeric polymer is ‘rubber-like’ in softness and flexibility processed as a thermoplastic material, are low-temperature toughness, stress-crack resistance. Other Rubber materials considered creating pressure points and complete insole.

The footwear system having pattern of the pressure points arranged in different zones to trigger the muscle/Neuron system of the wearer tested involving two group having 10 individuals each, one with group having absolutely normal routine with less stress parameters or within range of test requirements considered as Placebo Arm and second group marked as controlled and Third group as invention group both having regular pattern of stress in their routine work. The study designed on question-based investigation of wearer related to the experiencing about better feeling of health. The range of assessment rates were from 1 to 10 and allowed to wear for 15 days. The Normal group was given normal Footwear for assessment to deduct placebo effect from the controlled group. Placebo group observed the range from Zero effect to 2 ratings that ranges 0-2, for the first controlled the range observed 4 to 5 ratings whereas the invention group assessed 7 to 10 rating as the feeling of bitterness in their routine with detecting or monitoring system that helped the invention group to find out the diagnostic parameters that assist in improving health condition by adjusting their psychological and physiological parameters.

The invention relates to a system that includes an article of footwear with a sensor system as described above, with an electronic module connected to the sensor system, and an external device configured for communication with the electronic system/chip. The chip is configured to receive data from the sensors and to transmit the data to the external device, and the external device is configured for further processing the data.

Further, the present invention provides a footwear sensor which is able to detect changes in temperature around the foot of a wearer. Another object of the present invention is to provide a footwear sensor that is simple and easy to use. Invention provides a footwear sensor that is economical in cost to manufacture. The present invention is to provide a footwear sensor that is simple and easy to use.

Thus the embodiment of the present invention, a footwear having an inner sole that comprises a plurality of sensors positioned about an area of the inner sole for sensing conditions within the foot receiving portion and generating signals representative of the sensed conditions. A chip that connected to receive the generated signals from the plurality of sensors and analyses the signals to determine if a critical situation exists and monitoring the current health levels of the user. The sensors adopted in the present invention are chosen from Force, sensors adopted in the present invention are chosen from Force, accelerometer and Gyroscope. Accelerometers measure specific forces or accelerations, Gyroscope used for measuring or maintaining orientation and angular velocity determine an improved estimate of orientation under dynamic human movement conditions by using both accelerometer and gyroscope readings.

The embodiment of the present invention, having Communication Port designed to standardize the connection of peripherals to external device that responsible for communication between sensors to external device to measure the signals receives in the readable format of the end-user. The female end of the connector is referred to as a port much faster and more compatible.

Plurality of sensors are engineered to detect the forces exerted and having a communication port, connected to the sensors and designed to transmitting data detected by each sensor in a universally readable format which information is stored/collected in a “chip”

The chip is configured to receive data from the sensors and to transmit the data to the external device, and the external device is configured for further processing the data A chip is connected to receive the generated signals from the plurality of sensors and analyses the signals to determine if a critical situation exists and monitoring the current health levels of the user.

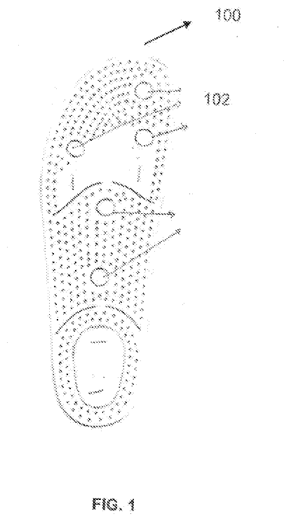

As illustrated in FIG. 1, the foot bed 100 comprises major pressure points 102. The zone near the toes serves the purpose for detecting activities pertaining to the brain, while the zone towards the middle, serves the purpose for detecting any fluctuations and abnormalities of digestive system while the zone near the heel that serves the purpose of enhancing painless long standing hours.

The cumulative effect of all three zones creating synergistic effects that enhance energy levels as well as blood circulation and pressure levels. These zones are as shown in the accompanying FIG. 1. The inner sole made to be reversed in case the wearer desires to not make use of such sensor systems.

As illustrated in FIG. 1, the zone near the toes serves the purpose for detecting activities pertaining to the brain, while the zone towards the middle, serves the purpose for detecting any fluctuations and abnormalities of digestive system while the zone near the heel that serves the purpose of enhancing painless long standing hours.

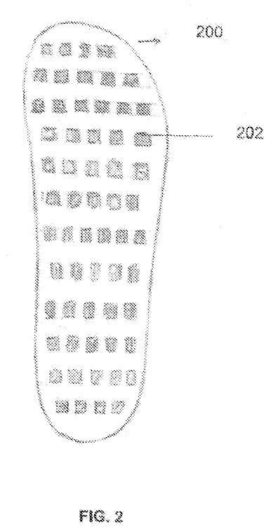

A sensory foot bed 200 is shown in FIG. 2. The sensory foot bed 200 has a sensor chip 202 for the purpose of collecting such data and converting it into a readable format for further analysis if needed. FIG. 2 illustrates a plurality of sensors 202 engineered to detect the forces exerted and having a communication port, connected to the sensors and designed to transmitting data detected by each sensor in a universally readable format which information is stored/collected in a “chip”.

According to one embodiment of the present invention, the sensor systems are present within the footwear, which consist of a plurality of sensors which are engineered to detect the forces exerted by the wearer of the footwear on the sensor. The footwear also has within it a communication port, connected to the sensors and designed in a manner which serves for transmitting data regarding the force exerted by the wearer of the footwear and detected by each sensor in a universally readable format which information is stored/collected in a “chip”.

According to one embodiment of the present invention, the following components are present in the digitalized therapeutic footwear:

i) NRF52840-QIAA-R7: Communication between shoes and mobile device is needed. This IC device has SOC with inbuilt Bluetooth feature and with very powerful communication instead of obstacles. This IC device has very high Memory 1 MB flash/256 KB RAM, all interfaces ADC, I²S, PDM, PWM, SPI, TWI, UART, USB, 48 multiple functions GPIO, small size, low cost, and is easily available.

ii) MX25R8035FM2IL0: Data has to be stored if in case communication loss between mobile app & shoes device happens. Considering for at least 3 days data back up the footwear is provided with 8 MB flash memory. The device has flash-nor topology with quad SPI interface. The device is in small size, low cost, and is easily available.

iii) MAX30102EFD+T: Sensors for needed for heart rate monitoring, body oxygen level & temperature measurement. This single IC has all three features.

iv) LSM6DSMTR: A sensor is needed for step count with direction. This IC has both features of accelerometer & Gyroscope with 6 axes.

v) HX711: Analog to digital conversion with high accuracy amplification is needed to measure weight of body. This IC has ADC amplification with high sensitivity.

vi) LG77LICMD: Tracker & Navigation are needed for live location. This module uses very low power support GNSS with band support of 1.561 GHZ, 1.575 GHz, 1.602 GHz. This is very small size, supports chip antenna which is suitable for wearable devices.

vii) TXS0102DCUR: This IC is used for level shifter as two devices are operating in two different supply voltage. This device is helpful to maintain appropriate voltage both sides.

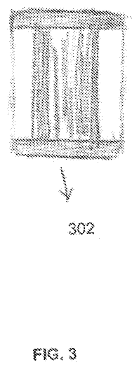

FIG. 3 illustrates a sensor chip 302. A chip is connected to receive the generated signals from the plurality of sensors and analyses the signals to determine if a critical situation exists and monitoring the current health levels of the user. The chip embedded in the footwear, thus serves the purpose to gather such information through the sensors, ultimately serves as a repository of any information gathered by the sensors.

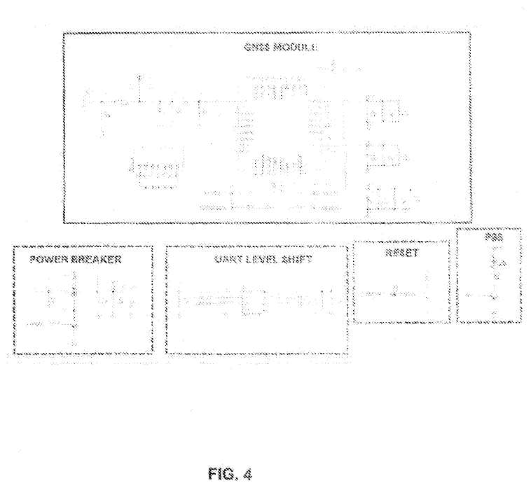

FIG. 4 illustrates schematic diagram of GNSS module, power module, UART level shift, reset and PSS.

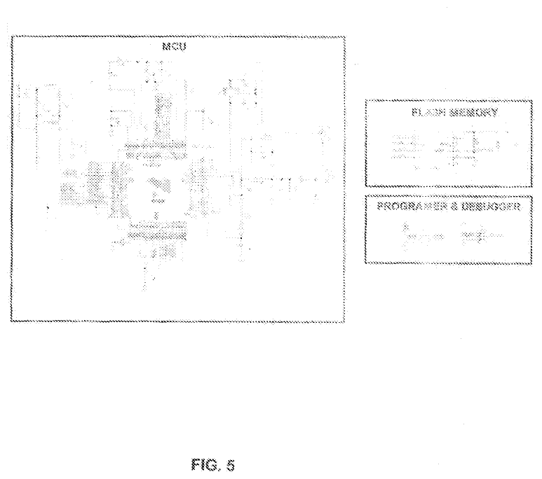

FIG. 5 illustrates schematic diagram of MCU, flash memory and programmer and debugger.

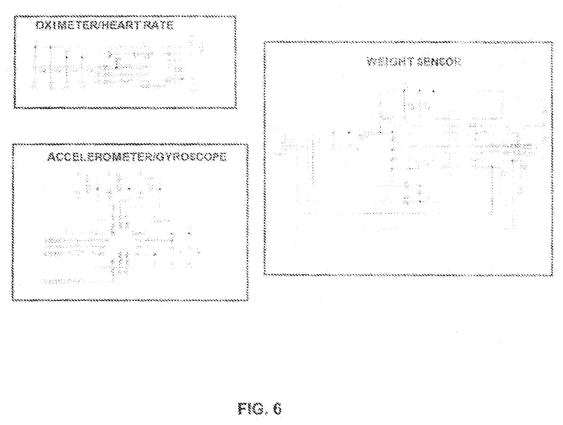

FIG. 6 illustrates schematic diagram of oximeter/heart rate, accelerometer/gyroscope and weight sensor

According to one embodiment of the invention, a digitalized therapeutic footwear for detecting ailments of health is disclosed. The digitalized therapeutic footwear comprises a plurality of pressure points, a sensor system, a data collector system attached to the inner parts footwear system, a communication port and an external computer device to receive data from data collector system. The pressure points transmit signal based on the condition of the foot to sensor system that is stored in data collector and sent to external computer device through communication port connected to the footwear.

The present invention relates to detecting ailments of health system provide early warning to ailments Related to brain, cardiac stem, Digestive system, enhancing long standing hours.

The present invention transmit the signals to device for detecting ailments of health system by analyzing the health diagnostic parameters including Heart rate, Pulse rate, Blood oxygen, blood pressure, ECG, Weight, Temperature, Number of steps, Calories, BMI, BMR, Body fat, Gait Analysis, Sleep Tracking, water. The assessment of these parameters alarm individual to take measures to adjust and adopt the better healthy workout and healthy life style. The detection of variation in said diagnostic parameters helps as prophylactic measure that avoids advancing of conditions to next stage.

The objective of invention to determine the GAIT Analysis including Average working speed, Swing/Stand Ratio, Step length, Step in angle, GAIT cycle, Step out angle, Event Interval. Gait analysis is an assessment of the way the body moves, usually by walking or running, from one place to another. The purpose of gait analysis is to detect any abnormalities in locomotion. The communication port is chosen from USB, C-USB ports connected to sensor system for transmitting data on force exerted by the wearer.

The present invention determines the health diagnostic parameters including Heart rate, Pulse rate, Blood oxygen, blood pressure, ECG, Weight, Temperature, Number of steps, Calories, BMI, BMR, Body fat, Gait Analysis, Sleep Tracking, water. The assessment of these parameters alarm individual to take measures to adjust and adopt the better healthy workout and healthy life style. The objective of invention to determine the GAIT Analysis including Average working speed, Swing/Stand Ratio, Step length, Step in angle, GAIT cycle, Step out angle, Event Interval.

It will be recognized that the above described subject matter may be embodied in other specific forms without departing from the scope or essential characteristics of the disclosure. Thus, it is understood that, the subject matter is not to be limited by the foregoing illustrative details, but it is rather to be defined by the appended claims.

While specific embodiments of the invention have been shown and described in detail to illustrate the novel and inventive features of the invention, it is understood that the invention may be embodied otherwise without departing from such principles.

I claim:

1. A digitalized therapeutic footwear for detecting ailments of health, the digitalized therapeutic footwear comprising:
a plurality of pressure points;
a sensor system;
a data collector system attached to the inner parts footwear system;
a communication port; and
an external computer device to receive data from data collector system characterized in that
the pressure points transmit signal based on the condition of the foot to sensor system that is stored in data collector and sent to external computer device through communication port connected to the footwear.

2. The digitalized therapeutic footwear as claimed in claim 1, wherein the sensor system comprises a plurality of sensors selected from force sensor, Gyroscope, accelerometer and combination thereof.

3. The digitalized therapeutic footwear as claimed in claim 1, wherein the pressure points prepared from the material selected from the group TPE, TPR, TPU, TPC, TPA, EVA and any other rubber moldable and combination thereof.

4. The digitalized therapeutic footwear as claimed in claim 1, wherein the shapes of pressure points are in the shape of pyramid, round, square or oval and combination thereof.

5. The digitalized therapeutic footwear as claimed in claim 1, wherein the Zone Pressure points comprising three zones, namely, Toe Zones, middle foot zone and heel zone thereof.

6. The digitalized therapeutic footwear as claimed in claim 1, wherein the Toe Zones Pressure points connects to detecting activities pertaining to the Brain.

7. The digitalized therapeutic footwear as claimed in claim 1, wherein the middle foot zone Pressure points connect to detecting activities pertaining to the digestive system.

8. The digitalized therapeutic footwear as claimed in claim 1, wherein the middle foot zone pressure points connect to detecting activities pertaining to the painless long standing.

9. The digitalized therapeutic footwear as claimed in claim 1, wherein the data collector system comprises a chip to store data in universally readable format.

10. The digitalized therapeutic footwear as claimed in claim 1, wherein the external device is configured to receive data from a mobile application configured to display readings on the screen of the device.
